(19) United States
(12) Patent Application Publication
(10) Pub. No.: US 2013/0110084 A1
(43) Pub. Date: May 2, 2013

(54) Devices and Methods for Processing a Biomaterial in a Closed System

(71) Applicants: Kimberly Ann Siems, Raleigh, NC (US); Sonya Olabisi Amelia Meheux Sherwood, Durham, NC (US); Krasimira Lambeva Aleksandrova, Hannover (DE); Lubomir Arseniev, Hannover (DE)

(72) Inventors: Kimberly Ann Siems, Raleigh, NC (US); Sonya Olabisi Amelia Meheux Sherwood, Durham, NC (US); Krasimira Lambeva Aleksandrova, Hannover (DE); Lubomir Arseniev, Hannover (DE)

(73) Assignee: CYTONET, LLC, Durham, NC (US)

(21) Appl. No.: 13/808,137

(22) PCT Filed: Jun. 30, 2011
(86) PCT No.: PCT/US2011/042510

§ 371 Date: Jan. 3, 2013

Related U.S. Application Data
(60) Provisional application No. 61/361,722, filed on Jul. 6, 2010.

(51) Int. Cl. A61M 5/14

(52) U.S. Cl. CPC A61M 5/1407
USPC 604/522; 604/264; 604/246; 604/500

(57) ABSTRACT

Disclosed herein are methods and devices for processing a biomaterial in a closed system and administering the processed biomaterial to a subject.

### CROSS-REFERENCE TO RELATED APPLICATIONS

This application claims the benefit of U.S. patent application Ser. No. 61/361,722, filed 6 Jul. 2010, which is herein incorporated by reference in its entirety.

### BACKGROUND OF THE INVENTION

1. Field of the Invention

The present invention generally relates to devices and methods for preparing, in a closed system, a biological composition.

2. Description of the Related Art

In modern medical science, therapeutic treatments often involve the use of biomaterials such as biologics. In this regard, various methods and devices are known for obtaining and preserving biomaterials for later use. For example, US 20050084838 discloses a device for collection and cryopreservation of a biological fluid, such as blood, bone marrow, or umbilical cord blood. In particular, US 20050084838 discloses a device for collecting the biological fluid from a donor and adding cryopreservatives thereto for long-term cryopreservation and storage.

Unfortunately, various chemicals are used to freeze the biomaterials, e.g. cells or keep the cells in a hibernation state, prior to using the cells for treatment of patients. Other unwanted materials, such as endotoxins, are often associated with biologics. Removal of such chemicals and unwanted materials is important for decreasing toxicity in patients as well as lowering the chances of an immunological reaction caused by the treatment. Current methods often dilute the composition containing the biomaterial in order to “reduce” the amount of the unwanted chemicals and other materials to the subject. Unfortunately, such methods also reduce the concentration of the biomaterial, which may result in a reduced therapeutic response. In addition, many current methods require the biomaterial to be processed in a sterile environment in order to prevent the biomaterial from being contaminated.

Thus, a need exists for the removal of unwanted chemicals and other materials from a composition comprising a biomaterial in a manner which can be performed under conditions which might not be sterile without contaminating the composition and diluting the concentration of the biomaterial.

### SUMMARY OF THE INVENTION

In some embodiments, the present invention provides devices for processing a biomaterial in a closed system, which can be a closed and sterile system, and delivering the processed biomaterial to a subject without introducing amounts of biological contaminants which may be deleterious and/or injurious to the biomaterial, the closed system, reagents used to process the biomaterial and/or the subject to be treated. As disclosed herein, the devices comprises a plurality of components having interior walls that define the closed system, said plurality comprising a first chamber and a second chamber in fluidic communication via at least one fluid line, at least one fluid line connector capable of being connected to at least one additional component without breaching the closed system. The devices may further comprise one or more additional components connected to the fluid line connector. In some embodiments, the additional component is a second fluid line connected to a third chamber. In some embodiments, the first chamber has an inlet port which may be connected to or capable of being connected to a third fluid line that is connected to an engagement. In some embodiments, the engagement is capable of being connected to a container which contains a first composition that comprises the biomaterial to be processed. The first composition may contain one or more undesirable ingredients. In some embodiments, the first chamber has an outlet port which is connected to or may be connected to a fourth fluid line that is connected to a delivery device. In some embodiments, the delivery device is capable of delivering the processed biomaterial to the subject. The fluid lines according to the devices of the present invention may comprise one or more fluid flow regulators, such as clamps, which are capable of sealing the fluid lines closed. One or more chambers may comprise one or more reagents for processing the biomaterial or adding to the processed biomaterial.

In some embodiments, the present invention provides methods for processing a biomaterial, in a closed system, and/or administering the processed biomaterial to a subject without introducing amounts of biological contaminants which may be deleterious and/or injurious to the biomaterial, the closed system, reagents used to process the biomaterial and/or the subject to be treated. In some embodiments, the closed systems, which may be closed and sterile systems, have a first chamber which has an inlet port and an outlet port; and a second chamber in fluidic communication via at least one fluid line; at least one fluid line connector capable of being connected to at least one additional component without breaching the closed system. Such methods may be conducted using a device in accordance with the present invention. The processing methods include adding the biomaterial to a closed system of the present invention by connecting a container containing a composition comprising the biomaterial to an inlet port of the closed system, contacting the composition with one or more reagents for processing the biomaterial, providing the processed biomaterial in a first chamber of the closed system, collecting any waste material in a second chamber of the closed system, isolating and separating the container which contained the composition comprising the biomaterial from the closed system, and isolating and separating the second chamber containing the waste material from the closed system. In some embodiments, the processed biomaterial may be delivered to the subject from the closed system by way of a delivery device connected to the outlet port. The delivery device may be a drug delivery device or may be attached to a drug delivery device. Such methods can be used to remove undesired ingredients from the composition and/or increase the concentration of the biomaterial in the composition to be delivered to a subject. In some embodiments, the biomaterials are cells such as stem cells, blood cells, or liver cells.

In some embodiments, the present invention provides kits for processing a biomaterial, in a closed system (which may be a closed and sterile system), and/or administering the processed biomaterial to a subject without introducing amounts of biological contaminants which may be deleterious and/or injurious to the biomaterial, the closed system, reagents used to process the biomaterial and/or the subject to be treated. In some embodiments, the present invention provides kits for processing a biomaterial, in a closed system, and/or administering the processed biomaterial to a subject comprising a plurality of components having interior walls that define the closed system packaged together with a delivery device capable of being connected to the closed system and delivering the processed biomaterial to the subject or being connected to a drug delivery device that delivers the processed biomaterial to the subject, and optionally one or more reagents for processing the biomaterial. Some kits may comprise a plurality of components having interior walls that define the closed system which are provided as two or more structures packaged together, which when connected to each other, form the closed system, and optionally one or more reagents for processing the biomaterial. Some kits may comprise a device in accordance with the present invention packaged together with one or more delivery device which are capable of being connected to the closed system and delivering the processed biomaterial to the subject or being connected to a drug delivery device that delivers the processed biomaterial to the subject, one or more reagents for processing the biomaterial, or both.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description serve to explain the principles of the invention.

### DESCRIPTION OF THE DRAWINGS

This invention is further understood by reference to the drawings wherein:

FIG. 1A schematically shows an exemplary device configuration according to the present invention.

FIG. 1B schematically shows the components of the device as set forth in FIG. 1A which form a closed system X, which may be a closed and sterile system, and device portion Y.

FIG. 1C schematically shows the system sections XA and XB which form the closed system X of the device as set forth in FIG. 1A. One or both system sections XA and XB may be closed and sterile systems.

FIG. 2 schematically shows an exemplary device configuration according to the present invention which may be used to process a composition comprising a biomaterial, such as human liver cells, and administer the processed biomaterial to a subject, such as a human.

### DETAILED DESCRIPTION OF THE INVENTION

The present invention generally relates to methods and devices for (A) processing, in a closed system (which may be a closed and sterile system), a biomaterial under conditions which may not be sterile, (B) administering, under conditions which may not be sterile, the processed biomaterial directly from the device, which contains the closed system (in which the biomaterial was processed), to a subject, such as a human subject, or to a drug delivery device that delivers the processed biomaterial received from the device to the subject, or (C) conducting both (A) and (B). In some embodiments, the methods and devices of the present invention allow the biomaterial to be processed without the introduction of one or more biological contaminants (which contaminants are deleterious to the biomaterial, any reagents which will be used to process the biomaterial, and the processed biomaterial) into the closed and sterile system. In some embodiments, the methods and devices of the present invention allow the processed biomaterial to be administered directly from the device having the closed system in which the biomaterial was processed to the subject or the drug delivery device without introducing, into the subject, one or more biological contaminants that are injurious to the subject.

As used herein, a “biomaterial” refers to a material that interacts with a biological system. Such biomaterials may be obtained from natural sources or synthesized using known chemical and biotech methods (e.g. recombinant DNA methods and cell cloning, etc.). In some embodiments, the biomaterial is a biologic. As used herein, a “biologic” is a material that has been created by a biological process which may occur in vivo, ex vivo or in vitro. The biologic may be made of sugars, proteins, nucleic acids, lipids, and the like, and can be whole cells (e.g. blood cells, stem cells, liver cells, etc.), biological fluids (e.g. plasma, cerebrospinal fluid, etc.), and tissues (e.g. whole organs and tissue explants) or preparations made therefrom (e.g. compositions that have been fortified and/or purified, compositions having additional ingredients, such as excipients and adjuvants, added thereto, etc.). As used herein, a “biological contaminant” refers to agents capable of causing a deleterious effect, such as putrefaction and/or fermentation, that is deleterious to (a) the given biomaterial to be processed, (b) any reagents that will be used to process the given biomaterial, and (c) the processed biomaterial, and agents that are capable of causing injurious effects in the subject, such as disease and/or infection, and/or capable of eliciting any undesired effect in the subject. In some embodiments, the biomaterial to be processed is a composition that comprises an ingredient, such as preservatives and cyroprotectants (e.g. dimethyl sulfoxide (DMSO), polyethylene glycol, amino acids, propanediol, etc.), which should be removed or reduced.

As discussed herein, “conditions which may not be sterile” include clinical settings (e.g. doctors' offices, operating rooms, ambulances, etc.) and non-clinical settings (e.g. non-medical buildings, places of residence, the outside environment, etc.). Such “conditions which may not sterile” may, in fact, be sterile or aseptic, but the sterile or aseptic state of the conditions under which the biomaterial is processed and/or administered to a subject is not known by the person(s) processing and/or administering the biomaterial and/or the subject.

As used herein, a “closed system” refers to the internal cavity of the device that is selectively isolated from the external environment by one or more walls of the components forming the device. As used herein, “selectively isolated” means that a desired substance may be actively introduced into the internal cavity and/or actively removed from the internal cavity without exposing the internal cavity to substances other than the desired substance. A closed system may be a closed and sterile system. As used herein, a “closed and sterile system” refers to a closed system that has been sterilized and/or a closed system that is substantially free of one or more biological contaminants. As used herein, a closed system that is “substantially free of biological contaminants” may contain one or more biological contaminants in amounts that do not generally result in deleterious and injurious effects.

As used herein, “processing” a biomaterial refers to one or more actions that are carried out on the biomaterial which results in: the biomaterial being purified, concentrated and/or isolated away from a material (e.g. a chemical or a biomolecule), a chemical and/or physical change to the biomaterial, the biomaterial being suitable for administration to the subject, or a combination thereof.

For example, the biomaterial may be purified by washing the biomaterial with a wash solution, such as a buffer, and then removing the spent solution from the closed and sterile system. The biomaterial may be concentrated by known centrifugation methods and/or a material associated with the biomaterial may be removed from the closed and sterile system. The biomaterial may be chemically and/or physically changed by introducing one or more reagents into the closed and sterile system and allowing the biomaterial and the reagent(s) to interact. The biomaterial may be made suitable for administering to a subject by mixing the biomaterial with an excipient.

### Device Components and Device Materials

In some embodiments, the closed systems of the present invention are defined by the walls of two or more components that are joined together to form all or part of the internal cavity. Such joints may be formed using methods and devices known in the art which result in the joint being hermetically sealed. In some embodiments, all of the joints of the device are hermetically sealed.

Devices according to the present invention comprise a plurality of chambers that are in fluidic communication with each other. As used herein, a “chamber” of the present invention refers to a structure in which a substance, such as a fluid and/or solid, may be contained therein for a desired period of time. In some embodiments, one or more chambers of the present invention have flexible walls. In some embodiments, one or more chambers are semi-rigid and/or rigid. The chambers of the present invention may be of any shape and size. In some embodiments, the shape and size of a given chamber is suitable for a given action to be performed on the chamber. For example, a chamber that will be subjected to centrifugation should have a shape and size which is suitable for using in a given centrifuge. Those skilled in the art may readily select shapes and sizes of chambers that are suitable for a given action or actions to be performed on the container. Examples of commercially available chambers include BLOOD-PACK® bags (Fenwal, Inc., Lake Zurich, Ill.); CRYOCYTE® bags (Baxter, Deerfield, Ill.); PEDI-PAK® Transfer Packs (Genesis BPS, Hackensack, N.J.); MINI-PLASCO® containers (B.Braun, Melsungen, Germany); and the like.

In some embodiments, a given chamber of a device may be in fluidic communication with some or all of the other chambers of the device. The fluidic communication may be direct or indirect. As used herein, “direct fluidic communication” between two chambers means that a fluid may flow from one chamber to the other chamber without first passing through an intervening chamber. As used herein, “indirect fluidic communication” between two chambers means that a fluid can flow from one chamber to the other chamber by passing through one or more intervening chambers (e.g. a plurality of chambers arranged in a series). In some embodiments, a first chamber may be in fluidic communication with a second chamber and the second chamber may be in fluidic communication with the first chamber and/or a third chamber. In some embodiments, the fluidic communication between the first chamber and the third chamber is a direct fluidic communication. In other embodiments, the fluidic communication between the first chamber and the third chamber is an indirect fluidic communication, i.e. a fluid from the first chamber must first flow through at least one intervening chamber, such as the second chamber and/or a fourth chamber, before reaching the third chamber, and vice versa.

The direct fluidic communication between two chambers may be by way of a fluid line which connects the chambers to each other or by way of the two chambers being directly connected, i.e. without the use of a fluid line, to each other. As used herein, a “fluid line” refers to a structure through which a fluid may flow. Although a fluid line is capable of containing a substance therein, such that a fluid line is a chamber, as defined herein, a “fluid line” refers to a structure through which a fluid flows that has a volume that is smaller than both the volume of the structure from which the fluid originates and the volume of the structure to which the fluid flows. In other words, a chamber has a larger volume than that of a fluid line connected thereto. One or more fluid lines in the devices of the present invention may be flexible and/or semi-rigid. The fluid lines of the present invention may have one or more desired cross-sectional shapes. In some embodiments, one or more fluid lines of the devices of the present invention have a round cross-sectional shape. In some preferred embodiments, one or more fluid lines are tubular in shape. In some preferred embodiments, one or more fluid lines are flexible tubing made of a biocompatible material. A fluid line, according to the present invention, may be an integral part of one or more chambers or a separate and distinct component that is joined to a chamber of the device. In some embodiments, a fluid line may branch into two or more secondary fluid lines.

In some embodiments, one or more fluid lines may be connected to a chamber and/or two or more fluid lines may be connected to each other. The connection may be a direct connection, e.g. one component directly connected to another component at a joint, or an indirect connection, e.g. a fluid line connector, between the components. As used herein, a “fluid line connector” is a structure that provides a sealed, preferably a hermetically sealed, connection between the connected components. A fluid line connector of the present invention may be a bidirectional connector, i.e. a connector that allows fluidic communication from a first component on a first side of the connector to a second component on a second side of the connector and vice versa, or a unidirectional connector, i.e. a connector that allows fluidic communication in only one direction, e.g. from a first side of the connector to a second side of the connector. In some embodiments, at least one fluid line connector may be a one-to-one connector, i.e. a fluid line connector that allows only two components to be directly connected to each other. In some embodiments, at least one fluid line connector may be a multi-connector, i.e. a fluid line connector which contains a plurality of connection points capable of connecting a plurality of components (e.g. one or more fluid lines and/or one or more chambers), wherein one or more components of the plurality of components may or may not be connected to one or more additional components in a serial fluidic circuit and/or a parallel fluidic circuit.

As used herein, a “serial fluidic circuit” refers to an arrangement of fluid lines alternating with chambers which are connected to each other so as to provide a single pathway through which a fluid flows through each chamber in succession. As used herein, a “parallel fluidic circuit” refers to an arrangement of fluid lines and chambers which are connected to each other so as to provide a plurality of pathways through which a fluid is capable of flowing from a first point. Examples of commercially available fluid lines and fluid line connectors include DISCOFIX® three-way stopcock valves (B.Braun, Melsungen, Germany); plasma transfer sets (Baxter, Deerfield, Ill.); Y-connectors with open injection sites (Genesis BPS, Hackensack, N.J.); and the like.

Some or all of the fluid lines may contain a fluid flow regulator. As used herein, a “fluid flow regulator” is structure that is capable of regulating the flow of a fluid. A fluid flow regulator may be capable of either allowing unrestricted fluid flow or completely preventing a fluid from flowing from one side of the fluid flow regulator to the other side of the fluid flow regulator. In some embodiments, a fluid flow regulator may restrict some, but not all of the fluid flow. In some embodiments, where the fluid flow regulator restricts some, but not all of the fluid flow, the fluid flow regulator may also be capable of allowing unrestricted fluid flow and/or completely preventing a fluid from flowing from one side of the fluid flow regulator to the other side of the fluid flow regulator.

A fluid flow regulator may be positioned within the internal cavity of the fluid line and may be provided as an integral part of the interior walls of the fluid line or as a separate and distinct component that is joined to the interior walls of the fluid line. Alternatively, a fluid flow regulator may be externally positioned along the fluid line and may or may not be removably attached to the exterior walls of the fluid line. A fluid flow regulator of the present invention may be a bidirectional regulator, i.e. a regulator that regulates fluid flow from a first component on a first side of the regulator to a second component on a second side of the regulator and vice versa, or a unidirectional regulator, i.e. a regulator that regulates fluid flow in only one direction, e.g. from a first side of the regulator to a second side of the regulator. A fluid flow regulator may be of any desired shape or size so long as it performs its desired function. Such shapes and sizes for a desired flow of fluid, or lack thereof, may be readily determined by those skilled in the art. In some embodiments, multiple fluid flow regulators may be placed on or in a given fluid line. In some embodiments, two fluid flow regulators are provided as a pair of regulators along a fluid line. The fluid flow regulators belonging to a pair of regulators may be of the same or different size, shape, material and/or type. Additionally, the pair of regulators may be positioned within the internal cavity of the fluid line or externally positioned. Alternatively, one fluid flow regulator may be positioned within the internal cavity of the fluid line and the other fluid flow regulator may be externally positioned along the fluid line. For example, a pair of fluid flow regulators may be placed in the fluid line 45.

A pair of fluid flow regulators may be used to isolate and/or separate a component, such as a chamber, from the closed system (a) without leaking or exposing the contents of the component to the external environment, and (b) without exposing the remaining closed system to the external environment by “sealing off” the part of the fluid line between the pair of fluid flow regulators by using both fluid flow regulators to prevent any fluid flow there between and then severing the fluid line between the pair of fluid flow regulators. Alternatively, one or more parts of the fluid line connecting the component to be separated and/or removed from the closed system may be permanently sealed, thereby “isolating” the component from the closed system, and optionally, the component may be “separated” from the closed system by severing the fluid line between two permanent seals or somewhere in the middle of a single permanent seal using methods and devices known in the art. In some embodiments, the formation of a permanent seal results in both isolation and separation of the component. For example, heat may be applied to a part of the fluid line such that the fluid line becomes heat sealed while a portion is melted away, thereby severing the fluid line while permanently sealing the ends of the fluid line that become disconnected. Examples of commercially available fluid flow regulators include slide clamps (Fenwal, Inc., Lake Zurich, Ill.); pinch clamps (Halkey-Roberts Corp., Saint Petersburg, Fla.); in-line stopcock valves; and the like.

The devices, according to the present invention, contain at least one port that allows access into and/or out of the closed system. In some embodiments, at least one port may be a one-to-one port, i.e. a port that allows only a single access point into and/or out of the closed system. In some embodiments, at least one port may be a multi-port, i.e. a port which contains a plurality of access points into and/or out of the closed system. In some embodiments, the devices contain at least one inlet port and at least one outlet port. As used herein, an “inlet port” is a structure through which a desired substance may be actively introduced into the closed system of the present invention without exposing the closed system to substances other than the desired substance. As used herein, an “outlet port” is a structure through which a desired substance may be actively removed from the closed system without exposing the closed system to substances other than the desired substance.

A port of the present invention may be a bidirectional port, i.e. a port that allows both access into and out of the closed system, or a unidirectional port, i.e. a port that allows access in only one direction, e.g. into the closed system. A port may be a multi-use port, i.e. a structure which allows repeated access into the closed system and/or repeated access out of the closed system. Alternatively, a port may be a single-use port, i.e. a structure that can be used only once to allow one-time access into and/or one-time access out of the closed system. For example, a single-use port may be permanently sealed closed by its use or after its use. A port, according to the present invention, may be provided as an integral part of a chamber or a fluid line. Alternatively, a port may be a separate and distinct component that is connected directly to a chamber or a fluid line at a joint or indirectly connected to the chamber or the fluid line via a fluid line connector. In some embodiments, a port may also be a fluid line connector, i.e. may be used as either a port or a fluid line connector which connects an additional component thereto. Examples of commercially available ports include sealed luer locks (Halkey-Roberts Corp., St. Petersburg, Fla.); resealing rubber septums; PEDI-PAK® Pedi-Syringe Filter™ devices (Genesis BPS, Hackensack, N.J.); and the like.

The devices of the present invention may include one or more engagements. As used herein, an “engagement” refers to a structure capable of forming a sealed, preferably a hermetically sealed, connection between a port of the device of the present invention with an apparatus external to the closed system. The engagement may be directly or indirectly (e.g. by way of a fluid line and/or a fluid line connector) connected to the port. Examples of commercially available engagements include spike adapters and spike-tube adapters (Origen Biomedical, Austin, Tex.); plasma transfer sets (Fenwal, Inc., Lake Zurich, Ill.); and the like. The apparatus external to the closed system may be a container from which a desired substance is introduced into and/or out of the closed system. In some embodiments, the apparatus external to the closed system is a drug delivery device which, when used, delivers the processed biomaterial to the subject. Any known drug delivery device may be used or adapted to be used in conjunction with the devices of the present invention using methods known in the art.

The components (i.e. chambers, fluid lines, fluid line connectors, fluid flow regulators, ports, and engagements) of the devices of the present invention may be made of plastics, glass, metals, and the like, and combinations thereof. In some embodiments, some or all of the components of the devices of the present invention are made of one or more materials which are biocompatible with a given biomaterial, reagents used to process the given biomaterial, the processed given biomaterial, and the subject to which the biomaterial is to be administered. Such materials are generally referred to as biocompatible materials. It is noted that the biocompatibility of a material depends on the particular biomaterial and reagents to be used and the subject to be treated. Nevertheless, those skilled in the art may readily determine, using methods and information known in the art, which materials are biocompatible materials for a given set of circumstances which circumstances include the particular biomaterial and reagents to be used and the subject to be treated, as well as the conditions under which the material will be exposed to such (e.g. time, temperature, concentration, etc.) and actions that will be performed on the material (e.g. heating, freezing, sterilizing, etc.). As used herein, a thing, such as a composition or a material, or a process conducted on the thing is referred to being “biocompatible” where the thing or the process does not have a toxic or injurious effect on a given biomaterial to be processed, reagents that will be used to process the given biomaterial, the resultant processed biomaterial, the particular subject to be treated, under the conditions of exposure thereto, and the actions to be performed thereon. In some embodiments, the methods and devices of the present invention and/or the methods and devices that are used in accordance with the present invention are biocompatible. A “biocompatible material” is a material that does not have a toxic or injurious effect on a given biomaterial, reagents that will be used to process the given biomaterial, the resultant processed biomaterial, the particular subject to be treated, under the conditions of exposure thereto, and the actions to be performed thereon. Such biomaterials may or may not meet one or more of the various biocompatibility standards as required by the U.S. Food and Drug Administration.

### Exemplary Device Configurations

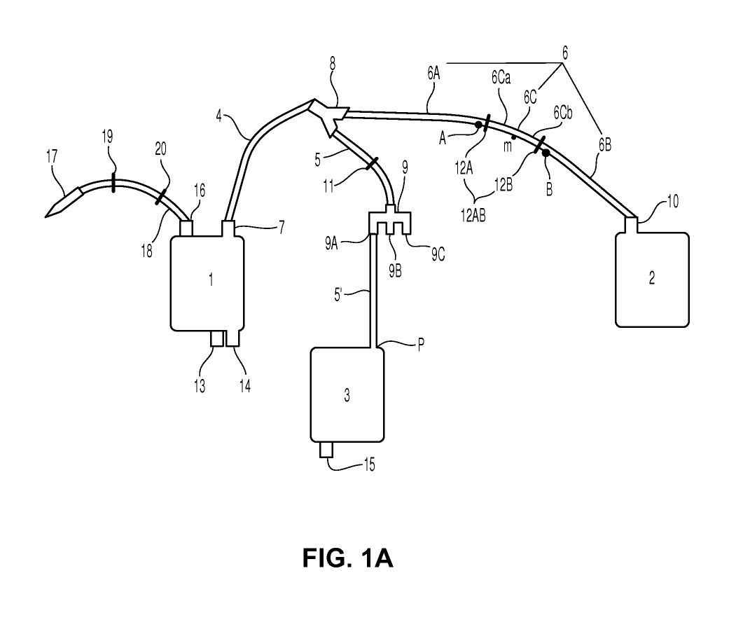

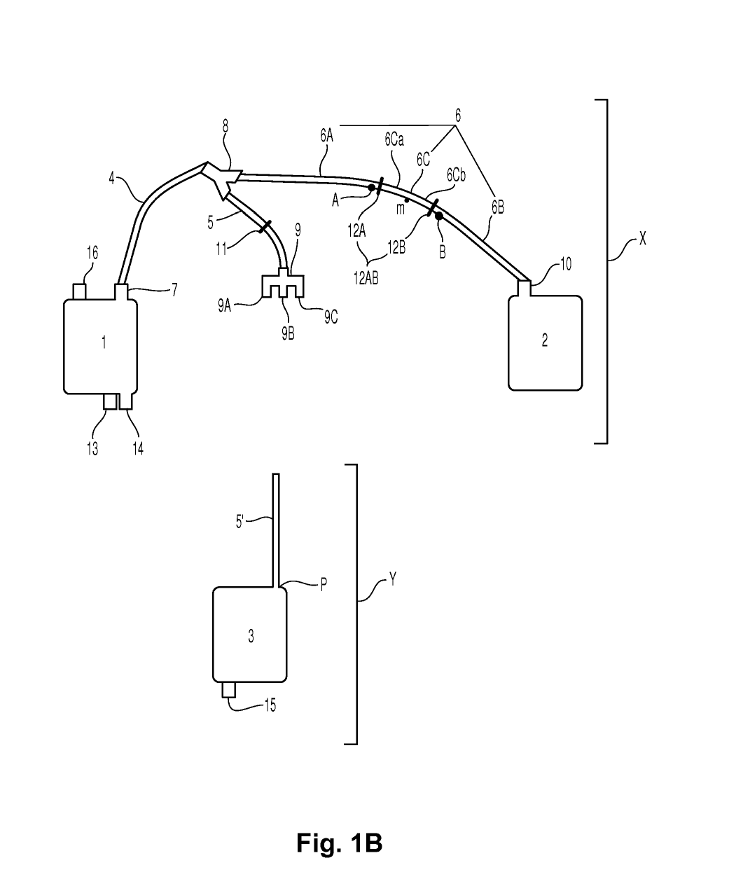

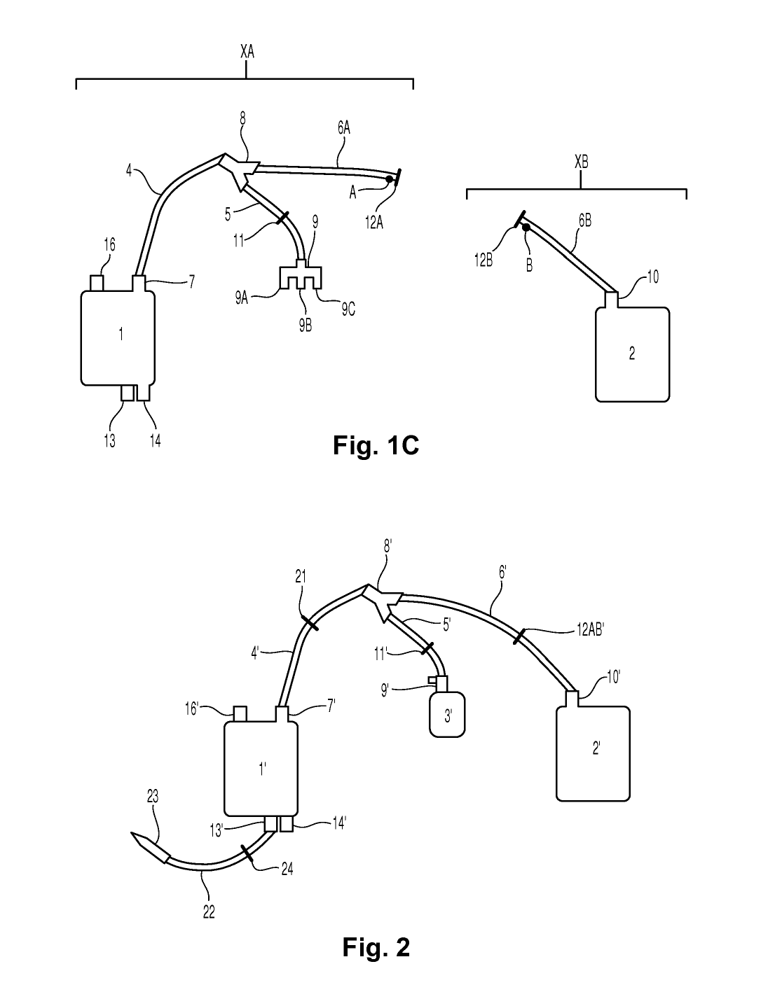

FIG. 1A schematically shows an exemplary configuration of a device according to the present invention. FIG. 1B shows the parts that form the closed system X, which may be a closed and sterile system, and the parts that form the device portion Y of the device of FIG. 1A. As shown in FIG. 1B, device portion Y comprises port 15 and fluid flow line 5′ that is directly connected to chamber 3 at joint P. In some embodiments, device portion Y may comprise additional components, e.g. another component may be attached at port 15. It is noted that fluid flow line 5′ may be of any length and may be optional (i.e. chamber 3 is directly connected to connection point 9A, B, or C, and may be indirectly connected to chamber 2).

As shown in FIG. 1B, the closed system X is an internal cavity defined by the interior walls of chambers 1 and 2, the interior walls of fluid lines 4, 5, and 6, the interior walls of fluid line connectors 7, 8, 9, and 10, the walls of fluid flow regulators (if positioned on the interior side of the fluid lines) 11, 12A and 12B, the interior walls of ports 13, 14 and 16. In some embodiments, the interior walls of fluid line 18 and engagement 17, as well as the walls of fluid flow regulators (if positioned on the interior side of the fluid lines) 19 and 20, may form a part of the closed system X (not shown).

As shown in FIG. 1B, fluid line 6 comprises fluid line sections 6A, 6B and 6C (composed of fluid line subsections 6Ca and 6Cb which are defined by an arbitrary point m). Fluid flow regulators 12A and 12B are provided as a pair of regulators. One or more of the fluid flow regulators (e.g. 11, 12A, 12B, 19 and 20) are optional. Thus, in some embodiments, instead of a pair of fluid flow regulators 12A and 12B, only one fluid flow regulator 12AB is provided. In some preferred embodiments, however, at least one fluid flow regulator is positioned on or in each fluid line between two components, e.g. chambers. Thus, in some embodiments, a fluid flow regulator is on or in fluid line 4. In other embodiments, in some embodiments, fluid line connector 8 and/or 9 is also a fluid flow regulator. In these embodiments, a separate fluid flow regulator is not necessary.

In some embodiments, a pair of regulators may be used to separate closed system X into system section XA and system section XB as shown in FIG. 1C. As shown in FIG. 1B, fluid flow regulators 12A and 12B may be used to separate system section XB (FIG. 1C), fluid flow regulator 12B, and fluid line subsection 6Cb away from the other components of the closed system without exposing system section XA (FIG. 1C) and/or system section XB (FIG. 1C) to the environment that is external to the closed system X by closing fluid flow regulators 12A and 12B and severing fluid line section 6C at an arbitrary point m. In some embodiments, fluid line section 6A may be heat-sealed closed at point A and fluid line section 6B may be heat-sealed closed at point B, and then the fluid line section 6C may be severed at an arbitrary point m. In some embodiments, only one fluid flow regulator may be used to isolate and/or separate system section XA from system section XB without exposing system section XA to the environment that is external to the closed system X.

As shown in FIG. 1C, the closed system X comprises system section XA and XB, both of which may be closed and sterile systems independent of each other. System section XA is the cavity defined by the interior walls of chamber 1, fluid lines 4 and 5, fluid line section 6A, fluid line connectors 7, 8 and 9 and ports 13, 14 and 16, and the wall(s) of fluid flow regulator (if positioned on the interior side of the fluid line) 12A which prevents fluid flow into or out of the end of fluid line section 6A that is connected to fluid flow regulator 12A. System section XB is the cavity defined by the interior walls of chamber 2 and the wall(s) of fluid flow regulator (if positioned on the interior side of the fluid line) which prevents fluid flow into or out of the end of fluid line section 6B that is connected to fluid flow regulator 12B, and the interior walls of any additional components connected to system section XB.

As shown in these figures, fluid line connector 7 is a one-to-one connector, fluid line connector 8 is a Y-type connector, fluid line connector 9 is a multi-connector having connection point 9A at which fluid line 5′ is connected, and connection points 9B and 9C to which one or more additional components may be connected, fluid line connector 10 is an integral part of chamber 2, and joint P is where fluid line 5′ is connected directly to chamber 3. Port 13 is an inlet and/or an outlet port (i.e. a unidirectional port or a bidirectional port) which may be a single-use port or a multi-use port, port 14 is an integral part of chamber 1, and port 15 may be used as a fluid line connector for connecting one or more additional components which may be provided as a serial fluidic circuit or as a parallel fluidic circuit. Port 16 may be a one-to-one port or a multi-port which provides a plurality of access points to which one or more engagements may be connected thereto.

In some embodiments, the devices of the present invention comprise one or more variations of the configuration as exemplified in FIG. 1A. Such variations include changing the position of a component illustrated in FIG. 1A. For example, port 16 may be provided on the bottom of chamber 1, fluid line 4 may branch into multiple fluid lines, the Y-type connector 8 may be absent and fluid lines 4 and 5 are directly connected to each other and fluid line 6 is connected to connection point 9B or 9C, a plurality of different engagements may be connected to port 16, and the like. Such variations also include changing the type of a component as illustrated in FIG. 1A. For example, the one-to-one port 16 may be changed to a multi-port having a plurality of access points that accommodate the connection of a plurality of engagements to the closed system, port 16 may be changed from a single-use port to a multi-use port inlet port 13, and/or port 16 may be changed to an outlet port, and the like. Such variations also include the addition of one or more additional components (e.g. chambers, fluid lines, fluid flow regulators, fluid line connectors, ports and engagements). For example, one or more additional fluid lines may be provided in the device and/or one or more additional fluid flow regulators (not shown) may be provided at any position on any one of the fluid lines of the device. Similarly, one or more additional fluid line connectors and/or one or more additional ports may be provided at any position on any one of the fluid lines and/or any one of the chambers of the device. In some embodiments, engagement 17, or, for example, one or more ports, such as an additional port on chamber 3, are configured so as to connect one or more external apparatuses, e.g. a syringe and an intravenous delivery device.

In some embodiments, the closed system X and device portion Y are separately provided to a downstream user to be connected. In some embodiments, the closed system X is provided to a downstream user as two portions which are then connected by the downstream user. In these embodiments, a first part (which comprises chambers 1 and 2, fluid lines 4, 5 and 6, fluid line connectors 7, 8 and 9, and ports 13, 14 and 16, and optionally fluid flow regulators 11, 12A and 12B) and a second part (which comprises fluid line 18, engagement 17, and fluid flow regulators 19 and 20). The components of the second part may be configured to deliver the processed biomaterial directly to a subject or configured to deliver the processed biomaterial to a drug delivery device which then delivers the processed material to the subject.

In some embodiments, chamber 1 is a processing chamber in which a biomaterial is contacted with one or more of reagents that may be contained in chamber 3 and/or introduced into chamber 1 via fluid line 4 and/or inlet port 13. In some embodiments, inlet port 13 is a multi-port having a plurality of access points which enable the introduction of a plurality of desired substances into the closed system X. In some embodiments, chamber 1 may be a chamber which is capable of being spun in a centrifuge. In some embodiments, chamber 1 may be removably attached to one or more sources, e.g. source containers, of the biomaterial to be processed. The sources may be the same or different. In some embodiments, a source container contains a pooled source, i.e. a mixture of different sources of the biomaterial to be processed.

In some embodiments, chamber 3 is a reagent chamber which contains one or more reagents for processing the biomaterial, where reagents may be separated from each other by, for example, one or more dividers which define separate compartments and each compartment may have its own fluid line which directly or indirectly connects it to chamber 1.

In some embodiments, chamber 2 is a collection chamber in which a material that has been separated from the biomaterial is collected. The material to be collected may be one or more spent reagents used to process the biomaterial and/or a compound composition that is naturally associated with the biomaterial in nature. In some embodiments, the material is discarded after collection. In other embodiments, the material is collected for subsequent use.

In some embodiments, the device comprises a closed system which is, at least, defined by a processing chamber in fluidic communication with a collection chamber. In some embodiments, the closed system is further defined by one or more fluid lines which provide the fluidic communication between the processing chamber and the collection chamber and at least one port which provides an access point into the closed system.

In some embodiments, the device further comprises one or more ports directly or indirectly connected to the processing chamber and/or one or more ports directly or indirectly connected to the collection chamber. In some embodiments, the device further comprises one or more reagents, such as buffers (e.g. Composol® PS (Fresenius Kabi, Bad Homburg v.d.h., Germany), preservatives, stabilizers, sterilizing agents, additives (e.g. glucose), and the like, for processing a biomaterial in processing chamber 1.

In some embodiments, devices of the present invention consist essentially of the components which define the closed system X. As used in this context, the transitional phrase “consists essentially of” means that the device may comprise additional components which do not materially change the closed system, i.e. prevent selective isolation of the internal cavity and/or cause loss of selective isolation of the internal cavity from its external environment (which is the environment at and/or surrounding the external surfaces of the walls of the plurality of components). Thus, in such embodiments, the plurality of components may further contain one or more additional components (i.e. chambers, fluid lines, fluid line connectors, fluid flow regulators, ports and engagements) that do not materially change the closed system.

In some embodiments, devices of the present invention comprise (1) a closed system which consists essentially of the components that form closed system X as schematically shown in FIG. 1B, and (2) a device portion which comprises one or more components that do not materially change the closed system X. In some embodiments, devices of the present invention comprise (1) a closed system which consists of the components that form closed system X as schematically shown in FIG. 1B, and (2) a device portion which comprises one or more components that do not materially change the closed system X. In some embodiments, devices of the present invention comprise (1) a part of a closed system which consists essentially of the components that form closed system X, as schematically shown in FIG. 1B, but without fluid line 18, engagement 17 and the flow regulators 19 and 20, and (2) a device portion which comprises one or more components that do not materially change the closed system X. In some embodiments, devices of the present invention comprise (1) a part of a closed system which consists of the components that form closed system X, as schematically shown in FIG. 1B, but without fluid line 18, engagement 17 and the flow regulators 19 and 20, and (2) a device portion which comprises one or more components that do not materially change the closed system X. In these embodiments, the device portion may comprise, consist essentially of, or consist of the components as schematically shown in FIG. 1B.

In some embodiments, the closed system of the present invention is defined by a plurality of components which consists of a processing chamber (which has one or more ports) having a direct connection or an indirect connection to a fluid line (which can be a first fluid line having a direct connection or an indirect connection to a second fluid line) that has a direct connection or an indirect connection to a collection chamber (which has one or more ports).

In some embodiments, a device of the present invention may be packaged in the form of a kit to then be assembled and used just prior to administering the processed biomaterial. For example, the plurality of components which are connected together to form a closed system according to the present invention may be packaged together with one or more components that may be used with the closed system. For example, the closed system X may be packaged together with additional device components, e.g. chamber 3 and fluid line 5′ having a small cavity which may contain a reagent for processing a given biomaterial. In some embodiments, one or more components for delivering the processed biomaterial is packaged together with the closed system according to the present invention.

### Methods for Constructing the Closed Systems

The closed systems of the present invention may be constructed using methods and devices known in the art. As disclosed herein, some or all of the steps for constructing a closed system of the present invention may be conducted under conditions which may not be sterile, for example, outside of a sterilized clean room. In some embodiments, one or more component members (i.e. components belonging to the plurality of components whose interior walls define a closed system in accordance with the present invention) may be formed from a commercially available system having multiple chambers that are in fluidic communication, individual components which may be commercially available and which are capable of being directly or indirectly connected to other components (that may or may not be member components), or a combination thereof. Commercially available systems having multiple chambers may be obtained from Fenwal, Inc. (Lake Zurich, Ill.).

The internal cavity that is formed by the interior walls of the multiple chambers that are in fluidic communication of an unmodified commercially available system is referred to herein as a “commercial cavity”. When the commercially available system is modified in such a way that causes a structural modification to the commercial cavity, the structurally modified commercial cavity is referred to herein as a “modified cavity”. A modified cavity may be a closed system in accordance with the present invention or the modified cavity may be one that is modified further in order to result in a closed system according to the present invention. In some embodiments, the modified cavity was modified by connecting one or more individual components such that one or more of the interior walls of the individual components defined part of the modified cavity.

If the commercially available system contains one or more component members that are undesired, e.g. the interior walls of the undesired component members should not define a portion of the modified cavity to be formed or be a part of the device to be formed, the undesired component members may be isolated and/or separated from one or more components. If the commercially available system contains too few components, one or more additional components may be directly or indirectly connected to one or more components of the commercially available system. In some embodiments, one or more interior walls of the additional components to be connected to one or more components of the commercially available system may or may not define a part of the resulting modified cavity. The methods and devices as described herein and/or methods and devices known in the art may be used to isolate, separate and/or connect such components.

In some embodiments, the methods and devices for isolating, separating and/or connecting components do not result in the introduction of amounts of one or more biological contaminants that generally result in deleterious and injurious effects into the original cavity and/or the modified cavity. For example, in some embodiments, a seal, such as a hermetic seal, and various sealing devices, such as thermal impulse sealers, heat sealers, sonic sealers, and others known in the art, are used to isolate, separate and/or connect various device components. An example of a commercially available sealing device is the Hematron III device (Fenwal, Inc., Lake Zurich, Ill.). An example of a commercially available device that can be used to connect components, e.g. two fluid lines, to be in fluidic communication without introducing significant amounts of biological contaminants is the sterile connecting device available from Terumo (Eschborn, Germany).

Any desired component that is capable of being sealed, as described herein, may be readily connected to one or more components, which may or may not be an original component of the commercially available system. Examples of components that can be added to the devices of the present invention include chambers having at least one extended portion that can be used to attach the chamber to another component, e.g. a single BLOODPACK® unit. Examples of extended portions that can be used to attach a component to another component include fluid lines which may be sealed at its free end, an extended access hub, and the like.

In some embodiments, all of the component members were individual components that were connected to each other such that the individual components are in fluidic communication and the individual components are connected such that one or more of the interior walls of the individual components form an internal cavity that is a closed system in accordance with the present invention.

In some embodiments, the closed system of the present invention is formed from one or more device portions and/or one or more cavity portions. As used herein, a “cavity portion” refers to a structure that forms or will form a portion of the internal cavity of the closed system. Such a structure can be one component or two or more components connected together. Similarly, a “device portion” refers to a structure that forms or will form a portion of the device excluding the internal cavity and the closed system. In some embodiments, a structure has a small cavity which is selectively isolated. In some embodiments, when a first structure of the cavity portion has a small cavity that is directly or indirectly connected to a second structure of the cavity portion which has a small cavity, the first and second structures are connected such that the small cavity of the first structure is combined with the small cavity of the second structure to form a closed system in accordance with the present invention. Methods and devices known in the art may be used to connect cavity portions and device portions. In some embodiments, methods and/or devices which are used to connect the structures of a cavity portion and/or two or more cavity portions do not result in the introduction of amounts of one or more biological contaminants that generally result in deleterious and injurious effects into the small cavities and/or the resulting closed system.

In some embodiments, where it is difficult or impossible to maintain a selective isolation of one or more small cavities and/or the closed system while constructing a device according to the present invention and/or adding or removing one or more components from the device, the steps which form the small cavities and/or resulting closed system may be conducted in a biological safety cabinet known in the art which provides an environment that is substantially free of biological contaminants.

In some embodiments, significant amounts of biological contaminants can be present in the closed system even when the selective isolation of the cavities (i.e. small cavities, original cavities, modified cavities, and internal cavities) has been maintained. Thus, in some embodiments, one or more of the cavities may be partially or completely sterilized using methods, devices and compositions known in the art, preferably the methods, devices and compositions employed herein are biocompatible.

### Processing and Treatment Methods

In some embodiments, the present invention is directed to methods of processing a biomaterial and treating a subject which comprises administering the biomaterial to the subject with a device of the present invention.

In particular, a biomaterial may be processed using the device as shown in

FIG. 1 or a variation thereof. For example, a biomaterial may be introduced into chamber 1 through port 13, 14, and/or the engagement of 17. One or more processing reagents may be added into chamber 1 from chamber 2, 3 and/or an additional chamber comprising of the reagent(s) which may be added to chamber 1 from connection point 9B or 9C or from port 13 or 14. When both the biomaterial and the processing reagents are in chamber 1, the biomaterial and the processing reagents may be mixed and after a given period of time and a given temperature, the mixture may be processed further. For example, additional reagents may be added thereto and/or the mixture may be subjected to centrifugation and the supernatant may be collected in chamber 2 or 3, or removed via ports 13 or 14 or connection points 9B or 9C. The biomaterial remaining in chamber 1 may be subjected to further washing and/or purification steps and/or one or more additional reagents may be added and the biomaterial may be resuspended therein. Chambers, e.g. chamber 2, containing collected material, e.g. spent wash solution and/or supernatant, may be removed as described herein.

In some embodiments, the biomaterial is added to chamber 1 in a manner that does not breach the closed system of the present invention. For example, the biomaterial may be in a container that has a delivery means that mates with an inlet port, e.g. port 13 or engagement 17, of the device of the present invention and forms a sealed, fluidic connection. In some embodiments, one or more of the processing steps for processing the biomaterial may be conducted in its original container and then added to chamber 1. In some embodiments, after the biomaterial to be processed is added to chamber 1, the original container may be removed, as described herein, such that the closed system is not breached.

In some embodiments, a sample of the biomaterial and/or processed biomaterial may be removed from the closed system for testing. In these embodiments, a port, e.g. port 13 or 14, may be used to remove the sample such that the closed system is not breached, as described herein. For example, in some embodiments, the port is made of a sealable plastic, a resealable plastic, or a self-healing plastic known in the art and the sample is removed using a sterile syringe. In some embodiments, the port is a sealed luer lock and the sample is removed using a syringe having a mating luer lock. In some embodiments, the port is a fluid line extension having its free end sealed where the sample can be removed by a needleless adaptor (such as that commercially available from Origen Biomedical, Austin, Tex.) connected thereto, or a portion of the fluid line extension having the sample therein may be removed using methods and devices known in the art (e.g. a heat sealer). In some embodiments, the sample may be removed by causing it to flow into another chamber connected to the closed system and then isolating the chamber containing the sample using the methods, as described herein, and then removing the sample using methods known in the art or separating the chamber from the closed system using the methods as described herein.

Once the biomaterial has been processed, the processed biomaterial may be directly or indirectly administered to a subject. For example, fluid line 18 and engagement 17 may be used to transport the processed biomaterial from chamber 1 to a drug delivery device which delivers the processed biomaterial to the subject. In some embodiments, a commercially available component such as a spike tube adapter (Origen Biomedical, Austin, Tex.) is used to connect chamber 1 to a drug delivery device that delivers the processed biomaterial to the subject. In some embodiments, a drug delivery device, such as a syringe, may be connected to port 13 or 14 or an additional access point on port 16, and then the biomaterial may be transferred from chamber 1 to the drug delivery device and administered to the subject.

In some embodiments, a second composition (e.g. a buffer solution, an additive, a drug, a biologic) may be added to the chamber containing the biomaterial such that both the processed biomaterial and the second composition are administered to the subject. In some embodiments, the second composition may be added to a second chamber that does not contain the processed biomaterial such that the processed biomaterial and the second composition can be separately administered to the subject or mixed together just prior to being administered to the subject.

It is noted that the processing methods and treatment methods of the present invention may be readily modified and optimized by those skilled in the art and that such modifications and optimizations may depend on a variety of factors which include the particular biomaterial to be processed, the subject to be treated, the mode of administration, and the like. Nevertheless, such modifications and optimizations are contemplated herein and considered to fall within the scope of the methods of the present invention so long as the biomaterial is processed by a device comprising a closed system, as described herein, and the processed biomaterial is then directly or indirectly administered to the subject using the device used to process the biomaterial.

In preferred embodiments, the closed system is a closed and sterile system and the processing steps and/or the treatment steps do not introduce amounts of biological contaminants that are deleterious and/or injurious to the biomaterial, the closed system, reagents used to process the biomaterial and/or the subject to be treated.

### Devices and Methods for Liver Cell Preparations

Particularly preferred embodiments of the present invention relate to devices and methods for processing liver cell compositions (as the biomaterial) and then administering the liver cell preparations (i.e. processed biomaterial) to a subject. The components and methods, as described herein, may be used to construct the devices for processing the liver cell compositions.

In these embodiments, a closed system according to the present invention comprises one or more reagents for processing a liver cell composition and the liver cell composition may be processed at the clinical site where the liver cell preparation will be administered to a subject.

FIG. 2 schematically shows a device configuration that is particularly preferred for processing liver cell compositions. As set forth in FIG. 2, the elements that are labeled with a prime (′) correspond to the elements as set forth in FIG. 2. It is noted that the devices for processing liver cell compositions according to the present invention may also comprise additional components, including one or more components as shown in FIG. 1A. As shown in FIG. 2, the device further comprises fluid flow regulators 21 and 24, fluid line 22, and engagement 23. In preferred embodiments, fluid line 22, engagement 23 and fluid flow regulator 24 are used to add the liver cell composition to chamber 1′ for processing. Engagement 23 may be a spike which forms a connection with the container having the liver cell composition to be processed.

In some embodiments, chamber 1′contains a buffer solution, e.g. Composol-PS®, for processing the liver cell compositions and chamber 3′ contains glucose for processing the liver cell compositions. In some embodiments, fluid line connector 9′ is a luer. In some embodiments, port 14′ is optional, chamber 2′ comprises one or more optional ports, and/or one or more additional fluid line regulators may be provided.

In some embodiments, a liver cell composition may be prepared for administration using the device as schematically shown in FIG. 2. In particular, a frozen human liver cell composition may be thawed and washed just prior to administering to a subject using the methods and devices described herein.

In particular, a bag containing a suspension of human liver cells may be thawed and washed as described below. This particular method may be readily modified by those skilled in the art for processing different types of cells, compositions, and amounts.

All solutions and the cell suspension after thawing are kept cool until the time of the administration.

A temperature controlled waterbath is set to an appropriately defined temperature for thawing cells. A refrigerated centrifuge is turned on and appropriately programmed such that the time is just long enough to separate the cells in the cell suspension.

Using the device of FIG. 2, fluid flow regulators 11, 12AB and 24, e.g. clamps, are employed to prevent fluid flow in lines 4′, 5′, 6′ and 22. Fluid flow regulator 11′ is opened to allow fluid flow from chamber 3′ containing glucose and the glucose is transferred through fluid lines 11′ and 4′ to chamber 1′ which contains Composol-PS®. A small amount of the contents of chamber 1′ are then passed through fluid lines 4′ and 11′ into chamber 3′ and then returned to chamber 1′ in order to rinse the fluid flow path and ensure the proper concentration of glucose in the mixture contained in chamber 1′. This mixture may be stored at 2-8° C. for a given period of time, i.e. 24 hours.

The liver cell composition is thawed in its container according to methods known in the art and as required for human administration. The container containing the thawed composition is connected to the device of FIG. 2 using engagement 23 and then the fluid flow regulator 24 is opened and the mixture in chamber 1′ is allowed to flow into the thawed container while continuously stirring gently. When about ⅔ of the mixture has flowed into the thawed container, the entire contents in the thawed container is transferred into chamber 1′ and the fluid flow regulator 24 is closed and the thawed container, engagement 23 and part of fluid line 22 is isolated and separated from the system such that the closed system is not breached.

Chamber 1′ is centrifuged to pellet the cells therein. Then most all of the supernatant is removed, i.e. just enough liquid remains to prevent the cell pellet from becoming too dry and unstable, by collecting the supernatant into chamber 2′. The cell pellet is then resuspended by gentle mixing and a sample for testing and counting is removed by isolating and separating chamber 3′ from the system such that the closed system is not breached. Chamber 2′ containing the supernatant is then isolated and separated from the system such that the closed system is not breached.

The cell suspension, i.e. liver cell preparation, in chamber 1′ is then administered to a subject directly or indirectly as described herein.

It is noted that the processing methods, in accordance with the present invention, can be used to remove undesired ingredients from the composition to be processed and/or increase the concentration of the biomaterial in the processed composition which is to be delivered to a subject without introducing amounts of biological contaminants which are deleterious and/or injurious to the biomaterial, the closed system, reagents used to process the biomaterial and/or the subject to be treated.

To the extent necessary to understand and/or complete the disclosure of the present invention, all publications, patents, and patent applications mentioned herein are expressly incorporated by reference therein to the same extent as though each were individually incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1. A device for processing a biomaterial in a closed system and delivering the processed biomaterial to a subject which comprises
a plurality of components having interior walls that define the closed system, said plurality comprising a first chamber and a second chamber in fluidic communication via at least one fluid line, at least one fluid line connector capable of being connected to at least one additional component without breaching the closed system.

2. The device according to claim 1, and further comprising the additional component connected to the fluid line connector.

3. The device according to claim 1, wherein the additional component is a second fluid line connected to a third chamber.

4. The device according to claim 1, wherein the first chamber has an inlet port.

5. The device according to claim 4, wherein the inlet port is connected to a third fluid line that is connected to an engagement.

6. The device according to claim 5, wherein the engagement is capable of being connected to a container which contains a first composition comprising the biomaterial to be processed.

7. The device according to claim 1, wherein the first chamber has an outlet port.

8. The device according to claim 7, wherein the outlet port is connected to a fourth fluid line that is connected to a delivery device.

9. The device according to claim 8, wherein the delivery device is capable of delivering the processed biomaterial to the subject.

10. The device according to claim 1, and further comprising at least one first fluid flow regulator on or in the fluid line.

11. The device according to claim 1, and further comprising at least one reagent for processing the biomaterial, said reagent is contained in the first chamber.

12. The device according to claim 1, and further comprising a second container containing the composition for processing the biomaterial or adding to the processed biomaterial.

13. A method for processing a biomaterial, in a closed system, for administering to a subject, wherein the closed system having a first chamber which has an inlet port and an outlet port; and a second chamber in fluidic communication via at least one fluid line; at least one fluid line connector capable of being connected to at least one additional component without breaching the closed system, which comprises:
a) adding the biomaterial to the closed system by connecting a container containing a composition comprising the biomaterial to the inlet port,
b) contacting the composition with one or more reagents for processing the biomaterial,
c) providing the processed biomaterial in the first chamber,
d) collecting any waste material in the second chamber,
e) isolating and separating the container which contained the composition comprising the biomaterial from the closed system, and
f) isolating and separating the second chamber containing the waste material from the closed system.

14. The method of claim 13, which further comprises administering the processed biomaterial to the subject by way of a delivery device connected to the outlet port.

15. The method of claim 13, wherein the delivery device is a drug delivery device or is attached to a drug delivery device.

16. The method of claim 13, wherein the composition comprises the biomaterial and at least one undesired ingredient and the processed biomaterial is a processed composition having a reduced concentration of the undesired ingredient and an increased concentration of the biomaterial as compared to the composition prior to processing.

17. The method of claim 13, wherein the biomaterial is liver cells.

18. A kit for processing a biomaterial, in a closed system, and/or administering the processed biomaterial to a subject comprising
a plurality of components having interior walls that define the closed system packaged together with
a delivery device capable of being connected to the closed system and delivering the processed biomaterial to the subject or being connected to a drug delivery device that delivers the processed biomaterial to the subject, and
optionally one or more reagents for processing the biomaterial.

19. A kit for processing a biomaterial, in a closed system, and/or administering the processed biomaterial to a subject comprising
a plurality of components having interior walls that define the closed system which are provided as two or more structures packaged together, which when connected to each other, form the closed system, and
optionally one or more reagents for processing the biomaterial.

20. A kit for processing a biomaterial, in a closed system, and administering the processed biomaterial to the subject comprising
the device according to claim 1 packaged together with a delivery device capable of being connected to the closed system and delivering the processed biomaterial to the subject or being connected to a drug delivery device that delivers the processed biomaterial to the subject, one or more reagents for processing the biomaterial, or both.

21. (canceled)

22. A processed biomaterial that was processed using a device according to claim 1.

23. A method for treating liver disease in a subject, which comprises administering to the subject a processed biomaterial having been processed using a device according to claim 1.

24. (canceled)

25. The method of claim 23, wherein the processed biomaterial is administered in a dosage form that is more concentrated and/or has less undesired ingredient(s) as compared to the corresponding unprocessed biomaterial.
